(19) United States
(12) Patent Application Publication
(10) Pub. No.: US 2005/0112190 A1
(43) Pub. Date: May 26, 2005

(54) Dosage form for controlled release of an active agent formulation

(76) Inventors: Lauren M. Wiser, Stanford, CA (US); Betty Yu, Mountain View, CA (US); Kimberly R. Capell, Fremont, CA (US); Andrew C. Lam, South San Francisco, CA (US); Zahedeh Hatamkhany, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/950,301

(22) Filed: Sep. 23, 2004

Related U.S. Application Data
(60) Provisional application No. 60/507,055, filed on Sep. 26, 2003.

(51) Int. Cl. A61L 15/16; A61K 9/48; A61K 9/22

(52) U.S. Cl. 424/451; 604/890.1

(57) ABSTRACT

The present invention is directed to a dosage form configured to provide the controlled release of an active agent formulation. A dosage form according to the present invention includes a reservoir containing an active agent formulation and an engine positioned at least partially within the reservoir. In order to reduce the possibility that the engine included in a dosage form of the present invention will separate from the reservoir either during or after fabrication of the dosage form of the present invention, the engine included in a dosage form according to the present invention is bonded to an inside surface of the reservoir. The present invention also includes methods for preparing a controlled release dosage form.

### CROSS-REFERENCE TO RELATED APPLICATIONS

This application claims benefit of U.S. Provisional Patent application No. 60/507,055, filed Sep. 26, 2003, which is incorporated herein by reference.

### BACKGROUND

1. Field of the Invention

The present invention relates to dosage forms capable of providing the controlled release of a variety of active agent formulations, including liquid active agent formulations. More specifically, the present invention is directed to a dosage form configured for the controlled release of an active agent formulation that includes a reservoir and an engine bonded to the reservoir, wherein the engine is formulated or configured to expel the active agent formulation from within the reservoir after administration of the dosage form.

2. State of the Art

Dosage forms providing controlled release of liquid active agent formulations are known in the art. For example, U.S. Pat. Nos. 5,245,357, 6,174,547, 5,830,502, and 5,614,578, U.S. Patent Publications numbered U.S. 2003-0198619 A1, U.S. 2003-0232078 A1, U.S. 2002-0071863A1, PCT Publications numbered WO 95/34285, WO 04/002448 and WO 01/41742, and PCT Patent Application numbered PCT/US04/24921 (not yet published), the contents of each of which are incorporated in their entirety herein by reference, disclose various different dosage form designs and active agent formulations suitable for providing dosage forms capable of delivering a liquid active agent formulation at controlled rate over a desired period of time. The benefits of controlled delivery of active agents are well recognized in the art, and dosage forms that achieve controlled delivery of liquid active agent formulations bring the benefits of controlled delivery to active agents that are not well suited to administration from conventional solid or tableted formulations.

As can be appreciated by references cited herein, dosage forms providing controlled release of liquid active agent formulations may be osmotically driven and created using reservoirs formed with various different hard or soft capsule materials. In addition, where a controlled release liquid active agent dosage form is osmotically driven, the osmotic engine included in such a dosage form may be coated on the outside surface of the reservoir or the osmotic engine may be encapsulated by the reservoir. Even further, as is taught in U.S. Patent Application Nos. 60/492,002 (PCT/US04/24921) and 60/392,774 (WO04/002448) (“the '002 application” and “the '774 application,” respectively), the osmotic engine may be only partly enclosed by the reservoir. Controlled release liquid active agent dosage forms that include engines that are positioned within the reservoir but are only partly encapsulated by reservoir forming material are presently thought to be advantageous. In particular, dosage forms that include an engine that is only partly encapsulated by the reservoir are thought to exhibit improved structural stability and more effectively preserve release rate functionality over time, especially where the engine included in the dosage form is an osmotic engine.

Despite the benefits provided by controlled release dosage forms that include an engine only partly encapsulated by the reservoir, dosage forms designed according to the teachings of the '002 application and the '774 application present manufacturing challenges. For example, the engine included in such dosage forms is positioned within the reservoir prior to one or more coating steps required to finish the dosage form. However, because the engine is held in place through a friction fit, the engine may be displaced or separated from the reservoir as pressure is exerted against the reservoir or the reservoir and engine are subjected to other mechanical stresses during the manufacturing process. Separation or displacement of the engine may be particularly problematic at commercial production scales, as the product batches are typically subjected to various mechanical stresses during automated production processes and the batch sizes are relatively large, which can magnify the stresses exerted against each dosage form due to the number and collective weight of the dosage forms included in each batch. Moreover, because the liquid active agent formulation may be loaded within the reservoir before placement of the engine, separation of the engine from the reservoir during subsequent manufacturing steps is particularly undesirable, as it not only results in the manufacture of a defective dosage form, but can also lead to the loss of active agent and contamination of an entire process batch.

It would be an improvement in the art, therefore, to provide a controlled release active agent formulation dosage form that offers the benefits achieved by dosage forms such as those taught in the '002 application and the '774 application, but is better suited to commercial scale manufacturing. Specifically, it would be an improvement in the art to provide a controlled release liquid active agent dosage form the includes an engine only partially encapsulated by the reservoir containing the active agent formulation but is designed to more effectively retain the engine at a proper position within the reservoir as the dosage form is manufactured. Ideally, the design of such a dosage form would not compromise release rate functionality and would allow the delivery of a wide range of active agent formulations at various different controlled rates.

### SUMMARY OF THE INVENTION

In one aspect, the present invention is directed to a dosage form configured to provide the controlled release of an active agent formulation. A dosage form according to the present invention includes a reservoir containing an active agent formulation and an engine positioned at least partially within the reservoir. The opening of the reservoir and the engine included in a dosage form of the present invention are sized and shaped such that the engine can be received within the opening and positioned such that at least a portion of the engine extends into the reservoir. Moreover, the engine and the reservoir are configured such that, once the engine is positioned within the opening of the reservoir, the osmotic engine is not completely encapsulated by the reservoir. The dosage form of the present invention is designed and configured in a manner that provides a dosage form that operates to expel the active agent formulation from within the reservoir at a controlled rate after administration of the dosage form to an environment of operation.

In order to reduce the possibility that the engine included in a dosage form of the present invention will separate from the reservoir either during or after fabrication of the dosage form of the present invention, the engine included in a dosage form according to the present invention is bonded to an inside surface of the reservoir. Bonding the engine of the dosage form of the present invention to an inside surface of the reservoir not only serves to reduce the frequency with which the engine separates from the reservoir, but, depending on the process used, the bond formed between the engine and the reservoir may provide a seal that works to reduce the likelihood that the active agent formulation included in the reservoir will leak from the reservoir by passing around the engine.

Various different materials and methods may be used to bond the engine to an inside surface of the reservoir. In one embodiment of the dosage form of the present invention, the engine is bonded using an adhesive material applied to an inside surface of the reservoir, to an outer surface of the engine, or to both an inside surface of the reservoir and an outer surface of the engine. In another embodiment, the engine is bonded to the reservoir by application of a solvent to an inside surface of the reservoir, to an outer surface of the engine, or to both an inside surface of the reservoir or an outer surface of the engine. In such an embodiment, the solvent works to solubilize reservoir forming material and material included on the engine such that, as the solvent dries, a bond between the reservoir and the engine is formed. In yet another embodiment, the engine is bonded to an inside surface of the reservoir using a heat sealing technique, such as tack or spot welding, laser welding, a hot wheel technique, or a heat facilitated crimping or clamping technique.

The engine included in a dosage form of the present invention can be any formulation, device or system that can be bonded to the reservoir and can function alone or in conjunction with other components of the dosage form to cause expulsion of the active agent formulation from within the reservoir at a controlled rate. For example, the engine included in a dosage form of the present invention may be an osmotic engine or other expandable formulation, device or system. Where the engine included in the dosage form of the present invention is an osmotic engine, the engine includes an expandable osmotic composition and may further include a barrier layer or an outer coating designed to limit migration of the active agent formulation into the osmotic engine.

In one embodiment, the dosage form of the present invention includes a reservoir containing an active agent formulation, an osmotic engine positioned within an opening formed within the reservoir, a rate controlling membrane, and an exit orifice through which the active agent formulation can be delivered. The rate controlling membrane is configured and formulated such that, upon administration of the dosage form to an environment of operation, water passes through the rate controlling membrane and into the osmotic engine at a controlled rate, which, in turn, results in the controlled expansion of the osmotic engine. As the osmotic engine expands, it extends into the reservoir and expels the active agent formulation from within the reservoir through the exit orifice at a rate that is proportional to the rate at which water passes into the osmotic engine through the rate controlling membrane.

The reservoir included in the dosage form of the present invention may be formed of any material suitable for use in a controlled release dosage form according to the present invention, and the material used to form the reservoir may vary as, for instance, the desired operational environment or composition of the active agent varies. In one embodiment, the reservoir is formed of a material that is permeable to water. In another embodiment, the reservoir is formed of a material that is substantially impermeable to water. In each embodiment, the reservoir may be formed of a single layer of material that provides desired performance characteristics, or, alternatively, the reservoir included in the dosage form of the present invention may be formed using multiple layers of one or more different materials.

In another aspect, the present invention is directed to a method of manufacturing a dosage form providing the controlled release of an active agent formulation. In each embodiment, the method of the present invention includes providing a reservoir having an opening that is sized and shaped to receive an engine, providing an engine, positioning the engine within the opening of the reservoir and bonding the engine to the reservoir. The step of bonding the engine to the reservoir can take place as the engine is positioned within the opening of the reservoir or after the engine has been positioned within the opening, as desired. The method of the present invention also includes loading an active agent formulation into the reservoir, and configuring the dosage form of the present invention such that an exit orifice is included or formed in the reservoir to allow delivery of the active agent formulation. Though the active agent is preferably loaded before the engine is positioned within and bonded to the reservoir, loading the active agent formulation in the dosage form of the present invention may also take place after the engine and reservoir have been operatively associated.

In one embodiment, the method of the present invention includes bonding the engine to the reservoir using an adhesive. In such an embodiment, the step of bonding may include applying an adhesive to an inside surface of the reservoir, an outside surface of the engine, or to both prior to positioning the engine within the opening of the reservoir. Alternatively, depending on the method used to apply the adhesive, the step of bonding may include applying an adhesive to an inside surface of the reservoir, an outside surface of the engine, or to both simultaneously with the step of positioning the engine within the opening of the reservoir.

In another embodiment, the method of the present invention includes bonding the engine to the reservoir using a solvent. In such an embodiment, the step of bonding may include applying a solvent to an inside surface of the reservoir, an outside surface of the engine, or to both prior to positioning the engine within the opening of the reservoir. Alternatively, depending on the method used to apply the solvent, the step of bonding may include applying a solvent to an inside surface of the reservoir, an outside surface of the engine, or to both simultaneously with the step of positioning the engine within the opening of the reservoir.

Were the step of bonding the engine to the reservoir includes the application of an adhesive or a solvent, bonding the engine to the reservoir may also take place after the engine has been positioned within the opening of the reservoir. In such an embodiment, the solvent or adhesive may be introduced into the interstitial spaces formed between an inside surface of the reservoir and an outside surface of the engine either through a passive mechanism, such as capillary action, or by forced introduction, such as by injection or by application of the solvent or adhesive in an environment pressurized to above atmospheric pressure.

In another embodiment, the method of the present invention includes bonding the engine to the reservoir using a heat sealing process. Where a heat sealing process is used, heat is applied to the engine, reservoir, or both such that material included in the engine, the reservoir, or in both the engine and reservoir is altered bonds the engine to the reservoir. The heat may be applied using any suitable process or mechanism, such as by tack or spot welding, laser welding, a hot wheel technique, or a heat facilitated crimping or clamping technique.

In yet another embodiment of the method of the present invention, the step of providing an engine includes providing an osmotic engine. Where the engine provided in the method of the present invention is an osmotic engine, the method of the present invention also includes providing a rate controlling membrane. Typically, the step of providing a rate controlling membrane includes forming or positioning a rate controlling membrane over at least the portion of the osmotic engine that is not encapsulated by the reservoir. Alternatively, depending on the type of material used to form the reservoir, the step of providing a rate controlling membrane may also include forming or positioning a rate controlling membrane over both the exposed portion of the osmotic engine and the reservoir.

In addition, where the engine provided in a method according to the present invention is an osmotic engine, the engine may be an osmotic engine that includes a barrier layer or is resistant to permeation by the active agent formulation. Where the engine provided is an osmotic engine including a barrier layer, the method of the present invention also includes orienting the osmotic engine before it is positioned within the reservoir such that after the engine is positioned within the opening of the reservoir, the barrier layer faces the active agent formulation. Proper orientation of an osmotic engine including a barrier layer within the reservoir is necessary to ensure operation of the engine and dosage form.

### BRIEF DESCRIPTION OF THE FIGURES

FIG. 1 through FIG. 6 provide schematic cross-sectional representations of different embodiments of the dosage form of the present invention.

FIG. 7 provides a graph illustrating the force required to expel an engine included in an exemplary intermediate dosage form prepared according to the present invention as well as the force required to expel an engine included in an intermediate dosage form that was not prepared according to the present invention.

FIG. 8 provides a graph illustrating the release rate performance of exemplary dosage forms according to the present invention as compared to controlled release liquid active agent dosage forms that do not include an osmotic engine bonded to the reservoir.

### DETAILED DESCRIPTION OF THE INVENTION

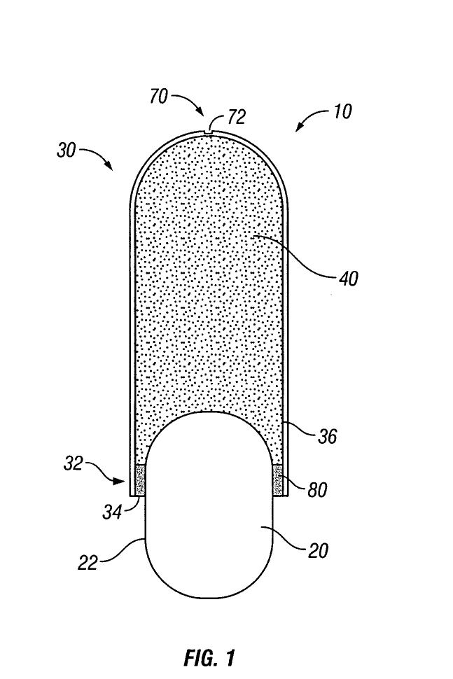

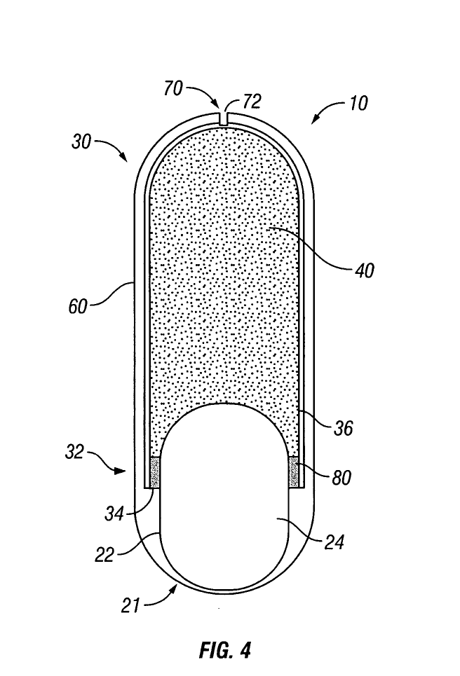

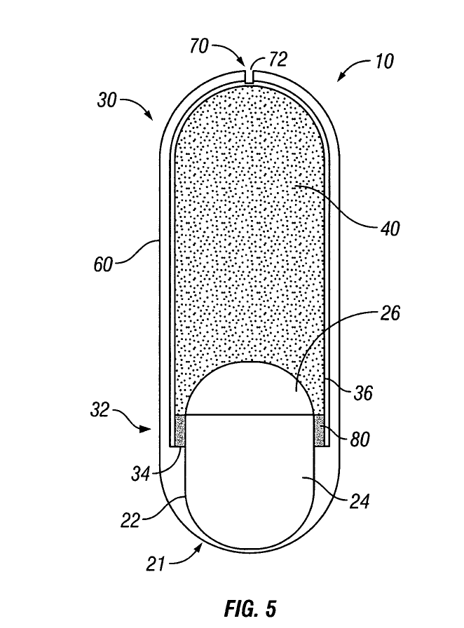

In one aspect, the present invention is directed to a dosage form. Various different embodiments of the dosage form 10 of the present invention are illustrated in FIG. 1 through FIG. 6. A dosage form 10 according to the present invention includes an engine 20 and a reservoir 30 suitable for containing an active agent formulation 40. The reservoir 30 and engine 20 are associated such that, as the dosage form 10 functions, the engine 20 operates to expel the active agent formulation 40 from within the reservoir 30 at a desired rate. In particular, the reservoir 30 of a dosage form of the present invention includes an opening 34 and the opening 34 of the reservoir 30 and engine 20 are sized and shaped to permit at least partial insertion of the engine 20 within the reservoir 30 through the opening 34 and boding of an outside surface 22 of the engine 20 to an inside surface 36 of the reservoir 30. As used herein, the terms “bond,” “bonded,” and “bonded to” refer to an engine that is associated with, such as by adhering, attaching, affixing, fastening, or otherwise joining to, a reservoir in a manner that increases the force required to displace the engine or dissociate the engine from the reservoir relative to an engine that is maintained within the reservoir solely by a friction fit.

The dosage form 10 of the present invention may be provided with any desired active agent formulation 40 that can be delivered from the dosage form 10. As it used herein, the expression “active agent” encompasses any drug, therapeutic compound, or composition that can be delivered to provide a benefit to an intended subject or environment. The expression “active agent formulation” is used herein to indicate a formulation that contains an active agent and can be discharged from a dosage form of the present invention as the dosage form operates in a desired environment of use. An active agent formulation 40 suitable for use in the dosage form 10 of the present invention is preferably a liquid formulation and may be neat liquid active agent or a solution, suspension, slurry, emulsion, self-emulsifying composition, liposomal composition, or other flowable formulation in which the active agent is present. The active agent formulation 40 may also be solid, or not flowable, before administration of the dosage form 10 to a desired environment of operation. However, where the active agent formulation 40 included in the dosage form 10 of the present invention is a solid formulation before administration, the formulation becomes flowable after administration. A solid active agent formulation may become flowable after administration due to, for example, the relatively higher temperature of the operational environment or the uptake of water into the active agent formulation.

A binder, antioxidant, pharmaceutically acceptable carrier, permeation enhancer, or the like may accompany the active agent in the active agent formulation 40. Further, the active agent formulation 40 may include a surfactant of mixture of surfactants. U.S. Pat. Nos. 5,245,357, 6,174,547, 5,830,502, and 5,614,578; U.S. Patent Publications numbered U.S. 2003-0198619, U.S. 2003-0232078, U.S. 2002-0071863; PCT Publications WO 04/002448, WO 95/34285, and U.S. Patent Application No. 60/492,002 (PCT/US04/24921), which are incorporated herein in their entirety by reference, detail exemplary drugs, carriers, and other constituents that may be used to form a active agent formulation 40 suitable for use in the dosage form 10 of the present invention.

The reservoir 30 included in a dosage form 10 of the present invention is formed to contain a desired amount of active agent formulation 40 and may be formed as desired to accommodate the engine 20. For example, the reservoir 30 can be formed with a first end 32 that includes an opening 34 that is sized and shaped to accommodate an engine 20 that operates to drive the active agent formulation from within the reservoir 30. Moreover, though the reservoir 30 of a dosage form 10 of the present invention may be formed in a generally oblong shape, the dosage form 10 according to the present invention is not so limited and may be manufactured to include a reservoir 30 that is sized and shaped as desired to suit a particular dosage form or active agent delivery application.

Though it may be formed in various shapes and sizes and includes an opening 34 designed to receive an engine 20, the reservoir 30 included in a dosage form 10 of the present invention does not completely enclose or encapsulate the engine 20. As is described in U.S. Patent Application Nos. 60/492,002 (PCT/US04/24921) and 60/392,774, (WO 04/002448) which are incorporated herein in their entirety by reference, designing a controlled release active agent dosage form to include a reservoir 30 that does not completely encapsulate the engine 20 can result in a dosage form that is easier to manufacture, exhibits improved structural stability, and better preserves release rate functionality. Moreover, designing a controlled release active agent dosage form to include a reservoir 30 that does not entirely encapsulate the engine 20 can facilitate the use reservoirs formed of a wider range of materials. For example, where the engine 20 included in a dosage form 10 of the present invention is an osmotic engine, the proper function of the engine 20 depends on an influx of water from an environment of operation. If the reservoir 30 is formed of a water impermeable material and is configured such that the reservoir 30 completely encloses the engine 20, the engine 20 could not function as desired to provide the controlled release of an active agent formulation 40.

The reservoir 30 included in a dosage form 10 of the present invention may be formed of a variety of materials. Any material that is compatible with a desired active agent formulation, is capable of being formed into a reservoir of desired shape and size, is suitable for administration to a desired environment of operation, and is capable of withstanding the anticipated storage conditions and operational stresses can be used to provide the reservoir 30 included in a dosage form 10 according to the present invention. Depending on the active agent formulation 40 included in the dosage form 10 and the desired performance characteristics of the dosage form 10, the reservoir 30 may be formed of a water permeable material or a material that is impermeable to water. A reservoir 30 useful in a dosage form according to the present invention may be fabricated by any suitable method. Examples of materials and methods that may be used to form a reservoir to be used in a dosage form 10 of the present invention are described in, for example, U.S. Pat. Nos. 6,183,466, 6,174,547, 6,153,678, 5,830,502, and 5,614,578, 5,245,357; U.S. Patent Publications Nos. U.S. 2003-0198619, U.S. 2003-0232078, U.S. 2002-0071863, PCT Publications WO 04/002448, WO 95/34285, and U.S. Patent Application No. 60/492,002 (PCT/US04/24921), the contents of each of which are incorporated herein by reference in their entirety.

Water permeable materials that may be used to form a reservoir 30 included in a dosage form 10 of the present invention include, for example, materials typically used to fabricate orally deliverable, liquid filled capsules. A water permeable reservoir 30 included in a dosage form 10 of the present invention may be formed using hydrophilic polymer materials or hydrophilic gelatin materials. Hydrophilic polymer materials, including cellulosic materials, provide preferred water permeable materials that may be used to form a reservoir 30 useful in a dosage form 10 of the present invention. Relative to the gelatin materials that are typically used in dosage form fabrication, water-soluble polymer materials are less susceptible to moisture loss and are less sensitive to changes in moisture content. As a result, a reservoir 30 formed using a hydrophilic polymer material may be better able to retain its structural integrity upon exposure to the active agent formulation 40 and the engine 20 included in a dosage form 10 of the present invention, particularly where the engine 20 is an osmotic engine 21 that exerts a high osmotic pressure. Moreover, because hydrophilic polymer materials are generally less susceptible to moisture loss, a reservoir 30 manufactured using hydrophilic polymer materials can be made such that less water is available to be drawn into the active agent formulation 40 from within the materials forming the reservoir 30 itself. Therefore, where a reservoir 30 of a dosage form 10 of the present invention is formed using a water permeable material, it is presently preferred that the water permeable material be formed of a hydrophilic polymer material.

Hydrophilic polymer materials that may be used to as the water permeable material included in a multilayer reservoir 30 include, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water permeable material included in a reservoir 30 of a dosage form 10 of the present invention may be manufactured using a single polymer material, the water permeable material may also be formed using a mixture of more than one polymer. Presently, because HPMC capsules for oral delivery of active agent formulations are commercially available and it has been found that capsule bodies formed of HPMC can be used to provide a reservoir 30 exhibiting suitable performance characteristics, the water permeable material included in a reservoir 30 of a dosage form 10 of the present invention is preferably formed using an HPMC material.

Where the reservoir 30 is formed of a material that is impermeable to water, the reservoir 30 can be made using a single material or a combination of materials. The material used to create a reservoir 30 that is suitable for use in a dosage form 10 according to the present invention and is impermeable to water according to the present invention need not be perfectly impermeable to the passage of water. As it is used herein, the term “impermeable” refers to reservoir formed of a material that exhibits a water flux of less than about 10⁻⁴ (mil·cm/atm·hr). Where the reservoir 30 included in a dosage form 10 of the present invention is formed using a water impermeable material, the water impermeable nature of the material serves to reduce or prevent migration of water from an external environment, through the reservoir 30, and into the active agent formulation 40.

In one embodiment, a water impermeable reservoir 30 suitable for use in a dosage form 10 according to the present invention is formed using a single layer of material that is impermeable to the passage of water. Materials suitable for forming such a reservoir 30 include, but are not limited to, water impermeable polymer materials. Where a single layer of water impermeable polymer material is used to form the reservoir 30, the polymer is preferably a synthetic resin or a combination of synthetic resins. Examples of water impermeable synthetic resins that may be used to form the reservoir 30 include, for example, linear polycondensation resins, condensation polymerized resins, addition polymerized resins, resins of phthalic anhydrides, polyvinyl resins such as polyethylene, polypropylene and their copolymers, polymer resins of methacrylic acid esters and acrylic acid esters, polycaprolactone, and copolymers of polycaprolactone with dilactide, diglycolide, valerolactone or decalactone. Different impermeable polymer materials and different combinations of impermeable polymer materials may be chosen to provide a reservoir 30 providing desired permeability, compatibility, and stability characteristics. A water impermeable reservoir may be formed, for example, using coating or molding techniques that are known in the art, such as, for example, those techniques described in U.S. Pat. Nos. 6,183,466, 6,153,678, 5,830,502, and 5,614,578 and in U.S. Patent Application Nos. 60/492,002 (PCT/US04/24921) and 60/392,774 (WO 04/002448), the contents of each of which are incorporated herein in their entirety.

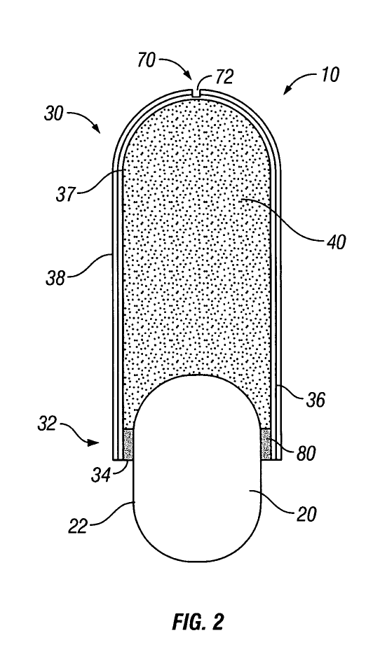

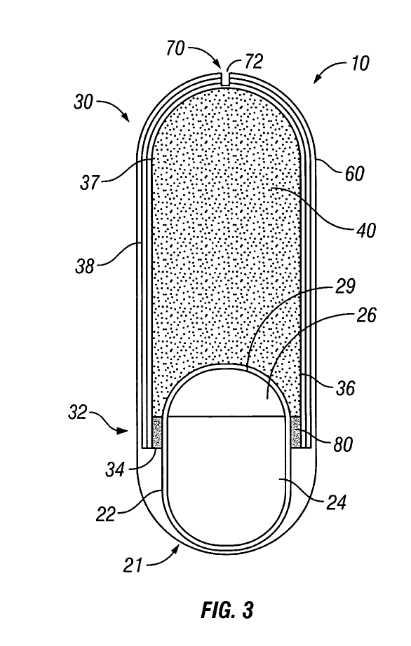

In an alternative embodiment, a water impermeable reservoir 30 included in a dosage form 10 according to the present invention may include two or more layers of different materials. For example, as is illustrated in FIG. 2 and FIG. 3, a reservoir 30 of a dosage form 10 of the present invention can include a water permeable material 37 coated with a water impermeable subcoat 38. The water permeable material 37 may be formed of a substance that is hydrophilic or otherwise permeable to the passage of water, such as the hydrophilic polymer and gelatin materials already described herein. The water permeable material 37 included in a water impermeable reservoir 30 included in a dosage form 10 according to the present invention may also be formed of a combination of water permeable and water impermeable materials. The water permeable material included in such a reservoir 30 may be formulated and formed by known methods, such as by the techniques described herein as useful in forming a water permeable reservoir 30 formed of a hydrophilic polymer or gelatin material. A water impermeable subcoat 38 included in a reservoir 30 of a dosage form 10 according to the present invention may be formed using any suitable water impermeable material that can be coated on or otherwise provided over the water permeable material 37. However, latex materials, such as Surelease® latex materials, which are available from Colorcon, Inc., Kollicoat ® SR latex materials, which are available from BASF, Eudragit® SR, and other polymethylacrylate latex materials, are presently preferred for forming a water impermeable subcoat 38. A water impermeable subcoat 38 may be provided over the water permeable material 37 included in a water impermeable reservoir 30 of a dosage form according to the present invention using any suitable coating or lamination technique. Coating processes suitable for providing a water impermeable subcoat 38 are described, for example, in U.S. Patent Application Nos. 60/492,002 (PCT/US504/24921) and 60/392,774 (WO 04/002448), the contents of which are incorporated in their entirety herein by reference.

The engine 20 included in the dosage form 10 of the present invention can be any composition, material, device or system that functions in an intended environment of operation to expel the active agent formulation from within the reservoir at a desired rate. For example, the engine 20 included in a dosage form 10 of the present invention may be an osmotic engine or other expandable formulation, device or system. After administration of the dosage form to an environment of operation, the engine 10 included in a dosage form of the present invention preferably operates by exerting a force against the active agent formulation 40 included in the reservoir 30 over a desired period of time, which force is sufficient to expel the active agent formulation 40 from within the reservoir 30.

In order to avoid any problems associated with permeation of the engine 20 by the active agent formulation 40 included in the dosage form 10, the engine 20 included in a dosage form 10 of the present invention is preferably resistant to permeation by the active agent formulation 40. As it is used herein, the terms “resistant to permeation” or “permeation resistant” refers to an engine that is configured or formulated such that, when included in a dosage form of the present invention, the engine exhibits an uptake of active agent formulation that is less than 5% by weight before administration of the dosage form. In preferred embodiments, the engine 20 included in the dosage form 10 of the present invention preferably exhibits an uptake of active agent formulation that is 3% by weight, or less, before administration of the dosage form, with engines exhibiting active agent formulation uptake of 1% by weight, or less, before administration of the dosage form being particularly preferred.

Though a dosage form 10 of the present invention may include any engine 20 capable of providing controlled release of an active agent formulation 40, the dosage form of the present invention is preferably fabricated with an osmotic engine 21. An osmotic engine 21 suitable for use in a dosage form 10 of the present invention includes an expandable osmotic composition 24 and is preferably prepared such that it is resistant to permeation by the active agent formulation 40 included in the dosage form.

An expandable osmotic composition 24 included in an engine 20 of a dosage form 10 according to the present invention may be formulated and formed using any materials and means that result in a composition that can be operatively associated with and bonded to the reservoir 30, is acceptable for the intended application of the dosage form 10, exhibits sufficient osmotic pressure to draw in water from an environment of operation over a desired period of time, and expands to exert a force sufficient to cause expulsion of an active agent formulation 40 from within a reservoir 30 as water is taken into the composition. The expandable osmotic composition 24 included in an osmotic engine 21 useful in a dosage form 10 of the present invention can be manufactured using known materials and methods, and may be formulated to provide an expandable osmotic composition 24 that is itself resistant to permeation by the active agent formulation 40 or can be made permeation resistant. Presently, the expandable osmotic composition 24 included in an osmotic engine 21 of a dosage form of the present invention is preferably formed as a tableted composition that includes a hydrophilic polymer capable of swelling or expanding upon interaction with water or aqueous biological fluids.

The expandable osmotic composition 24 included in an osmotic engine 21 used in a dosage form of the present invention may further include an osmotic agent, or “osmagent,” to increase the osmotic pressure exerted by the expandable osmotic composition 24, a suspending agent to provide stability and homogeneity to the expandable osmotic composition 24, a tableting lubricant, an antioxidant, or a non-toxic colorant or dye. Materials and methods that can be used to form an expandable osmotic composition 24 suitable for use in an osmotic engine 21 useful in a dosage form 10 of the present invention are taught, for example, in U.S. Pat. Nos. 6,174,547 and 6,245,357; U.S. patent publication Nos. U.S. 2003-0198619, U.S. 2003-0232078, U.S. 2002-0071863; PCT publications numbered WO 95/34285, WO 04/002448, and U.S. Patent Application No. 60/492,002 (PCT/US04/24921); the contents of each of which are herein incorporated in their entirety by reference.

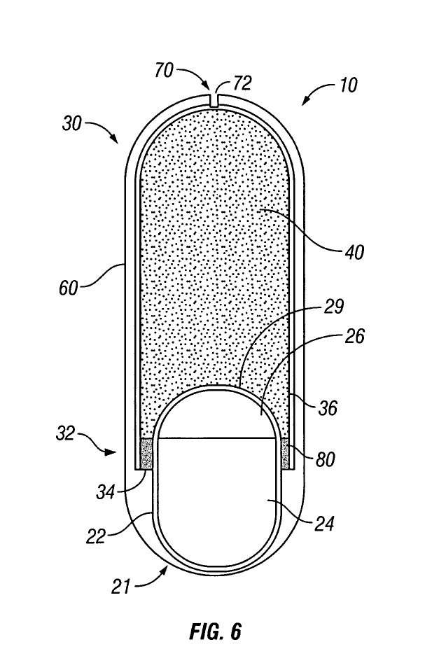

An osmotic engine 21 included in a dosage form of the present invention may also include a barrier layer 26. A barrier layer 26 included in an osmotic engine 21 used in a dosage form 10 according to the present invention is formulated of composition that is substantially impermeable to the active agent formulation 40. The barrier layer 26 works to reduce permeation of the expandable osmotic composition 24 by the active agent formulation 40. In addition, the barrier layer 26 serves to increase the uniformity with which the driving power of the expandable osmotic composition 24 is transferred to the active agent formulation 40. Where an osmotic engine 21 included in a dosage form 10 of the present invention includes a barrier layer 26, the barrier layer 26 and expandable osmotic composition 24 may be formed as a bi-layer tablet 28. Materials and methods suitable for creating such a bi-layer tablet 28 are taught, for example, in U.S. patent publication Nos. U.S. 2003-0198619, U.S. 2003-0232078; PCT publications numbered WO 95/34285, WO 04/002448, and U.S. Patent Application Nos. 60/492,002 (PCT/US04/24921); the contents of which are incorporated in their entirety herein by reference. Materials suitable for forming a barrier layer 26 useful in an osmotic engine 21 used in a dosage form 10 according to the present invention include, but are not limited to, a polymeric composition, a high density polyethylene, a wax, a rubber, a styrene butadiene, a calcium phosphate, a polysilicone, a nylon, Teflon®, a polystyrene, a polytetrafluoroethylene, halogenated polymers, a blend of a microcrystalline, high acetyl cellulose, or a high molecular weight fluid impermeable polymer.

Where desired, an osmotic engine 21 included in a dosage form 10 of the present invention may be a permeation resistant engine 20. A permeation resistant osmotic engine 21 useful in a dosage form 10 of the present invention may include an expandable osmotic composition 24 that is formulated to be permeation resistant as defined herein. However, where the expandable osmotic composition 24 included in an osmotic engine 21 according to the present invention is formed of a tableted, hydrophilic polymer composition, the expandable osmotic composition 24 will typically require further processing in order to render the expandable osmotic composition resistant 24 to permeation by an active agent formulation 40. For example, as is shown in FIG. 3 and FIG. 6, the expandable osmotic composition 24 may be provided with a permeation resistant coating 29 over at least an area of the expandable osmotic composition 24, wherein the coating 29 is formulated to be resistant to permeation by a given active agent formulation 40.

The materials used to form a permeation resistant coating 39 included in a permeation resistant osmotic engine 21 useful in a dosage form 10 of the present invention will vary depending on the nature of the active agent formulation 40 to which the expandable osmotic composition 24 must be made permeation resistant. In particular, to render the expandable osmotic composition 24 resistant to permeation by a hydrophobic active agent formulation, a permeation resistant coating 39 provided over the expandable osmotic composition will typically be a hydrophilic coating that is substantially impermeable to the hydrophobic active agent formulation. Alternatively, to render the expandable osmotic composition 24 resistant to permeation by a hydrophilic active agent formulation, a permeation resistant coating 39 provided over the expandable osmotic composition will typically be a hydrophobic coating that is substantially impermeable to the hydrophilic active agent formulation. As used herein, “substantially impermeable” refers to a coating composition that is sufficiently impermeable to an active agent formulation to render the expandable osmotic composition permeation resistant as defined herein. A permeation resistant coating 39 may be formulated using a variety of different naturally derived or synthetic materials, with materials and methods suitable for provide an permeation resistant osmotic engine being detailed in U.S. Patent Application No. 60/492,002 (PCT/04/24921), the contents of which are incorporated herein in their entirety by reference.

Where desired, a permeation resistant coating 39 may be formulated using blends of materials that provide desirable coating characteristics. For example, in order to achieve a permeation resistant coating 39 having desirable coating characteristics, it may be necessary to formulate the coating material using blends of film forming materials. In addition, a permeation resistant coating 39 according to the present invention may include one materials, such as a plasticizer, that improve the coating characteristics provided by a film forming material or a blend of film forming materials. In particular, where HPMC is used to form a permeation resistant coating 39 included in a permeation resistant engine useful in a dosage form 10 of the present invention, it is presently preferred that the HPMC coating is formulated using a plasticizer, such as PEG 8000. Importantly, a permeation resistant coating 39 is preferably formulated such that tensile strength of the permeation resistant coating 39 can be overcome by the force exerted by the expandable osmotic composition 24 as the engine 20 functions and the expandable osmotic composition 24 expands.

Where an engine 20 included in a dosage form of the present invention includes a permeation resistant coating 39 that is permeable to the passage of water, such as a coating that includes a hydrophilic polymer or water soluble component, the permeation resistant coating 39 may completely encapsulate the material or mechanism forming the engine 24. A permeation resistant coating 39 that encapsulates the expandable osmotic composition 24 included in an osmotically driven engine is formulated to exhibit a water permeability that is sufficient to permit water to enter the expandable osmotic composition 24 at a rate that allows the engine 20 to expand as needed to provide a desired release rate of active agent formulation 40. Moreover, if desired, where a permeation resistant coating 39 is provided over an osmotic engine, the thickness and water permeability of a permeation resistant coating 39 may be adjusted to provide a further measure of control over the release characteristics of a dosage form incorporating a permeation resistant engine 20. For example, in order to delay delivery of an active agent formulation 40 from a dosage form that incorporates an engine 20 having a permeation resistant coating 39 that encapsulates an expandable osmotic composition 24 and is permeable to water, the thickness of permeation resistant coating 39 may be increased until a desired delay is achieved.

However, a permeation resistant coating 39 included over an engine 20 included in a dosage form of the present invention need not entirely encapsulate the engine 20. In fact, where a permeation resistant coating 39 is included over an osmotic engine 21 and the permeation resistant coating 39 is impermeable to water or is not sufficiently permeable to water to allow the osmotic engine 21 to function as desired, the permeation resistant coating 39 is configured such that the permeation resistant coating 39 does not entirely encapsulate the expandable osmotic composition 24 including in the osmotic engine 21. In that manner, the water can be taken up by the expandable osmotic composition 21 at a rate that enables the osmotic engine 21 to function as desired.

An osmotic engine 21 included in a dosage form 10 of the present invention can be configured to include a barrier layer 26 and a permeation resistant coating 39. Moreover, where an osmotic engine 21 includes both a permeation resistant coating 39 and a barrier layer 26, the barrier layer 26 may be included within the permeation resistant coating 39 or on an outside surface of the permeation resistant coating 39. Materials and methods for fabricating an osmotic engine 21 that includes both a barrier layer 26 and a permeation resistant coating 39 are described in U.S. Patent Application No. 60/492,002 (PCT/04/24921), the contents of which are incorporated herein in their entirety by reference.

The engine 20 included in a dosage form 10 of the present invention is bonded to the reservoir 30 containing the active agent formulation 40. In particular, and outside surface 22 of the engine 20 is bonded to an inside surface 36 of the reservoir 30. Such bonding can take place as the engine 20 is positioned within the opening 34 formed in the reservoir 30 or after the engine 20 is positioned within the opening 34. However, in order to reduce the possibility that the engine 20 will be partially or entirely displaced from its desired position within the reservoir 30, where the engine 30 and reservoir 30 are bonded after the engine is positioned within the opening of the reservoir 32, the bonding step preferably takes place before any other processing steps are undertaken to complete the dosage form 10.

The engine 20 of the dosage form 10 of the present invention is bonded to an inside surface 36 of the reservoir 30 using a bonding material 80. As used herein, the term bonding material includes and substance useful for creating a bond as defined herein between the engine 20 and the reservoir 30 of the dosage form 10 of the present invention. The bonding material 80 included in a dosage form of the present invention may be applied to or introduced between the engine 20 and the reservoir 30 to form the desired bond. Alternatively, the bonding material 80 may include material used in fabricating the engine 20 or the reservoir 30 themselves.

In one embodiment, the bonding material 80 included in a dosage form 10 of the present invention is an adhesive. Any adhesive that is non-toxic in the desired environment of operation, provides a bond between the engine and the reservoir that is sufficiently strong to maintain the engine within the reservoir during manufacture of the dosage form, and is compatible with remaining components of the dosage form may be used in a dosage form 10 according to the present invention. As it is used herein, the term “compatible with” refers to adhesives that do not significantly compromise the stability or functionality of the remaining components of the dosage form 10, including the engine 20, the reservoir 30, and the active agent formulation 40. Where an adhesive is used to bond the engine 20 to the reservoir 30, the adhesive may be applied prior to, during, or after positioning the engine 20 within the opening 34 formed in the reservoir 34. Where the adhesive is applied after the engine 20 is positioned within the reservoir 30, the adhesive will typically exhibit a viscosity and surface tension that allows the adhesive to be taken up between the outside surface 22 of the engine and an inside surface 36 of the reservoir 30, such as by capillary action.

Adhesives suitable for use in a dosage form 10 of the present invention include naturally and synthetically derived materials. Examples of adhesives that may be used to bond the engine 20 to the reservoir 30 of the dosage form 10 of the present invention include, but are not limited to, naturally derived animal materials, such as albumin animal glue, casein, shellac, beeswax, naturally derived plant materials, such as oils, resins, waxes, rubbers, carbohydrates, gum Arabic, tragacanth, colophony, balsam, carnauba wax, linseed oil, and plant-derived proteins, starches, and dextrins, inorganic and mineral materials, such as silicates, magnesia, phosphates, litharge, and sulfur containing materials, synthetically derived materials, such as synthetic elastomers, synthetic rubbers, butyl, polyisobutylene, polybutadiene blends, polyisoprenes, polychloroprene, polyurethane, silicone, polysulfide, and polyolefins, thermoplastic materials and cellulose derivatives, such as acetate, acetate-butyrate, caprate, nitrate, methyl cellulose, hydroxyl ethyl cellulose, ethyl cellulose, carboxy methyl cellulose, vinyl polymers, such as polyvinyl acetate, polyvinyl alcohol, and polyvinyl chloride, polyester materials, such as polyesters, polystyrenes, and polyamides, polyacrylate materials, such as methacrylate and acrylate polymers, cyanoacrylates, polyether materials, such as polyhydroxyether and polyphenolic ethers, polysulfone materials, thermosetting amino plastics, such as urea and melamine formaldehydes, epoxy materials, such as epoxy polyamide, epoxy bitumen, epoxy polysulfide, and epoxy nylon, phenolic resins, such as phenol and resorcinol formaldehydes, phenolic-nitrile, phenolic-neoprene, and phenolic-epoxy, unsaturated polyester materials, polyaromatic materials, such as polyimides, polybenzimidazole, and polyphenylene, and furane materials, such as phenol furfural.

In another embodiment, the bonding material 80 used in a dosage form 10 of the present invention is formed using a solvent. Where a solvent is used to form the bonding material, the solvent is chosen such that it solubilizes a material included on the inside surface 36 of the reservoir 30 as well as a material included on an outside surface 22 of the engine 20. Therefore, as the solvent is introduced at an interface between the engine 20 and the reservoir 30 material from both the reservoir 30 and the engine 20 is dissolved, mixes and forms a bonding material 80. As the solvent dries, the mix of dissolved engine and reservoir forming material dries and fuses as a mix of material that bonds the engine 20 to the reservoir 30. In a preferred embodiment, the solvent used to form the bonding material dissolves a sufficient amount of reservoir forming material and engine forming material that the bonding material formed is substantially continuous with the engine 20 and the reservoir 30 and a substantially continuous bond is formed between the engine 20 and the reservoir 30.

Any suitable aqueous or organic solvent may be used to form the bonding material 80 included in a dosage form 10 of the present invention. Presently, purified water is a preferred solvent for forming the boding material 80. Alcohols, such as ethanol, are also presently preferred solvents for forming the bonding material 80. Even further, the solvents used to form the bonding material 80 may be a combination of solvents or a solvent system including two or more solvents, such as two or more organic solvents, two or more aqueous solvents, or a combination of one or more aqueous solvents with one or more organic solvents.

The use of a solvent to form the bonding material 80 works particularly well where the engine 20 included in the dosage form 10 of the present invention is coated with a material that is the same as, substantially similar to, or exhibits comparable solubility characteristics to the material forming the inside surface 36 of the reservoir 30. In one embodiment, the dosage form 10 of the present invention includes a reservoir 30 formed of a water soluble cellulosic material, such as HPMC, and an engine 20 coated with a water soluble cellulosic material, such as HPMC or another polymer material having similar solubility characteristics. In such an embodiment, the bonding material can them be formed by introducing a solvent, such as water or ethanol, or a combination of solvents, such as a mixture of water and ethanol, into an area where the outside surface 22 of the engine 20 interfaces with an inside surface 36 of the reservoir 30.

The adhesive or solvent applied to form the bonding material 80 included in a dosage form 10 of the present invention may be applied using methods or processes known in the art. For example, where the adhesive or solvent is applied before the engine 20 is positioned within the opening 34 of the reservoir 30 or as the engine 20 is positioned within the opening 34 of the reservoir 30, the adhesive or solvent may be applied by spraying the engine 20 with the desired adhesive or solvent, passing the engine 20 over or through a sponge or other applicator that transfers the adhesive or solvent to an outer surface 22 of the engine 20, or dipping the engine 20 in the adhesive or solvent used to form the bonding material 80. Alternatively, where the solvent or adhesive is applied after the engine 20 is positioned within the opening 34 of the reservoir 30, the solvent or adhesive may be applied at the interface formed between the opening 34 of the reservoir 30 and the outer surface 22 of the engine 20 by any suitable means that allows the solvent or adhesive to be drawn up, such as by capillary action, between the engine 20 and an inner surface 36 of the reservoir 30. In addition, where the solvent or adhesive is applied after the engine 20 is positioned within the opening 34 of the reservoir 30, the solvent or adhesive may be actively disposed between the inside surface 36 of the reservoir 30 and the outside surface 22 of the engine, such as by injection or by introduction of the solvent or adhesive at the interface formed between the opening 34 of the reservoir 30 and the outer surface 22 of the engine 20 in an environment pressurized above atmospheric pressure.

In yet a further embodiment, the engine 20 of the dosage form 10 of the present invention is bonded the reservoir 30 using a heat sealing technique. In such an embodiment, the reservoir 30, the engine 20, or both the reservoir 30 and engine 20 include heat responsive material that forms a bond between the engine 20 and reservoir 30 as heat is applied. The heat responsive material may be formulated to melt to form a bonding material that, upon cooling, fuses the engine 20 to the reservoir 30. Alternatively, the heat responsive material may be formulated to shrink either during or after the application of heat in a manner that bonds the engine 20 to the reservoir 30. Even further, the heat responsive material may be physically altered in any other fashion, such as softening, as heat is applied to form a more intimate interface between the engine 20 and reservoir 30 so that, upon cooling, the engine 20 is bonded to the reservoir 30. A variety of heat responsive materials suitable for use in forming the bonding material 80 are known in the art, and include heat responsive polymer materials.

Where a heat sealing technique is used to form the bonding material 80 included in the dosage form 10 of the present invention, the heat sealing material may be provided by the engine 20, the reservoir 30, or both. In one embodiment, the engine 20 is coated with a heat responsive material that serves as the bonding material 80 and creates a bond between the engine 20 and reservoir 30 upon application of a suitable heat sealing technique. In another embodiment, the inside surface 36 of the reservoir includes a heat responsive material that serves as the bonding material 80 and creates a bond between the engine 20 and reservoir 30 upon application of a suitable heat sealing technique. In yet another embodiment, the reservoir 30 is formed using a heat responsive material that serves as the bonding material 80 and creates a bond between the engine 20 and reservoir 30 upon application of a suitable heat sealing technique. Suitable heat sealing techniques that may be used to bond the engine 20 of a dosage form 10 of the present invention to the reservoir 30 of the dosage form 10 include, but are not limited to, known tack, spot, or laser welding techniques, hot wheel techniques, or a heat facilitated crimping or clamping techniques.

Regardless of the particular materials or methods used to bond the engine 20 to the reservoir 30 of the dosage form 10 of the present invention, bonding the engine 20 to the reservoir 30 reduces the likelihood that the engine 20 will be displaced from a desired position or separated from the reservoir 30 during further processing steps. Moreover, depending on the material and the method used to bond the engine 20 to the reservoir 30, the bond formed between the engine 20 and the reservoir 30 may work to more effectively seal the interface between the engine 20 and the reservoir 30 from penetration by the active agent formulation 40. Therefore, bonding the engine 20 to the reservoir 30 not only provides a physically more robust controlled release active agent dosage form that is better suited to commercial production, but can also provide a dosage form that is less susceptible to the undesirable loss or leaking of active agent formulation from within the reservoir.

Where the dosage form of the present invention includes an osmotic engine 21, the dosage form 10 preferably includes a rate controlling membrane 60. A rate controlling membrane 60 included on a dosage form 10 of the present invention allows water or aqueous fluid from the desired environment of operation to enter the osmotic engine 21 at a controlled rate and thereby facilitates controlled expansion of the osmotic engine 21 and controlled delivery of the active agent formulation 40 from the dosage form 10. A rate controlling membrane 60 included in a dosage form 10 according to the present invention is non-toxic in the intended environment of operation and maintains its physical and chemical integrity during the operation of the dosage form 10. Adjusting the thickness or chemical make-up of the rate controlling membrane 60 can control the rate at which the expandable osmotic composition 24 included in an osmotic engine 21 expands after the dosage form 10 is administered. Therefore, a rate controlling membrane 60 included in a dosage form 10 of the present invention that utilizes an osmotic engine 21 serves to control the release rate or release rate profile achieved by a dosage form 10.

A rate controlling membrane 60 for use in a dosage form 10 of the present invention may be formed using any material that is permeable to water, is substantially impermeable to the active agent, is pharmaceutically acceptable, and is compatible with the other components of the dosage form 10 of the present invention. Generally, a rate controlling membrane 60 will be formed as a semipermeable membrane using materials that include semipermeable polymers, semipermeable homopolymers, semipermeable copolymers, and semipermeable terpolymers. Semipermeable polymers are known in the art, as exemplified by U.S. Pat. No. 4,077,407, which is incorporated herein by this reference, and they can be made by procedures described in Encyclopedia of Polymer Science and Technology, Vol. 3, pages 325 to 354, 1964, published by Interscience Publishers, Inc., New York. A rate controlling membrane 60 included in the dosage form 10 of the present invention may also include a plasticizer to impart flexibility and elongation properties to the rate controlling membrane 60 or a flux regulating agent, such as a flux enhancing or a flux reducing agent, to assist in regulating the fluid permeability or flux through the rate controlling membrane 60.

A rate controlling membrane 60 included in a dosage form 10 according to the present invention is provided over at least the portion 27 of an osmotic engine 21 that is not enclosed or encapsulated by the reservoir 30. If desired, a rate controlling membrane 60 included in a dosage form 10 of the present invention may also be provided over both the reservoir 30 and the exposed portion 27 of the osmotic engine 21. Moreover, where a dosage form 10 according to the present invention includes a reservoir 30 that is permeable to water, a rate controlling membrane 60 included in the dosage form 10 preferable extends over both the reservoir 60 and the exposed portion 27 of the osmotic engine 21.

Methods for providing a rate controlling membrane 60 suitable for use in a dosage form 10 according to the present invention are known in the art and include any suitable coating technique, such as a suitable dip coating or spray coating process. Additional references describing materials and methods suitable for fabricating rate controlling membranes suitable for use in a oral dosage form 10 of the present invention include, for example, U.S. Pat. Nos. 6,174,547 and 6,245,357; U.S. patent publication Nos. U.S. 2003-0198619, U.S. 2003-0232078, U.S. 2002-0071863; PCT publication Nos. WO 95/34285, WO 04/002448, and U.S. Patent Application No. 60/492,002 (PCT/US04/24921), the contents which are incorporated in their entirety herein by reference.

A dosage form 10 according to the present invention also includes an exit orifice 70. The exit orifice 70 may include any structure, device, or dosage form configuration that allows active agent formulation 40 to be delivered from the reservoir 30 of the dosage form. An exit orifice 70 included in a dosage form 10 of the present invention may be embodied by one of various different structures. For example, the exit orifice 70 may include an aperture 72 formed partially or completely through the wall of the reservoir 30 included in the dosage form 10. Alternatively, as is shown in FIG. 3 through FIG. 6, where the dosage form 10 of the present invention includes a rate controlling membrane 60 over the reservoir 30, the exit orifice 70 may include an aperture 72 formed through the rate controlling membrane 60, or the exit orifice may include an aperture 72 formed through a rate controlling membrane 60 and a portion of the reservoir, such as a water impermeable subcoat 58 included in a reservoir 30 formed of multiple material layers. An exit orifice 70 formed of an aperture 72 may be formed by any suitable means, such as by suitable mechanical or laser drilling technologies.

Though the aperture 72 illustrated in FIG. 1 through FIG. 6 does not pass entirely through the reservoir 30 included in the dosage form 10, the aperture 72 allows the formation of an exit orifice as the dosage form is placed within or begins to operate within an intended environment of operation. In particular, where a dosage form 10 of the present invention includes a reservoir 30 formed of a single layer of water impermeable material, the aperture 72 formed in the rate controlling membrane 60 creates a breaking point where the material forming the reservoir 30 is compromised as the engine 20 included in the dosage form 10 begins to function and pressure within the reservoir 30 builds. Alternatively, where a dosage form 10 of the present invention includes a water permeable material and the aperture 72 exposes such material to the environment of operation, the water present in the environment of operation can work to weaken or dissolve the exposed portion of the reservoir 30, allowing the active agent formulation 40 contained within the reservoir 30 to be expelled as the engine 20 operates.

Nevertheless, the dosage form 10 of the present invention is not limited to an exit orifice 70 formed by an aperture 72. Where desired, the exit orifice may include an aperture that passes completely through the reservoir. Again, mechanical or laser drilling technologies may be used to create such an exit orifice. However, where the exit orifice provided in the dosage form of the present invention is formed through the reservoir, a closure sealing the exit orifice be needed. Any one of several means may be employed to provide such a closure. For instance, the closure may include a layer of material that covers the exit orifice and is arranged over a portion the outer surface of the dosage form, or the closure may include a stopper, such as a bung, cork, or impermeable plug, or an erodible element, such as a gelatin plug or a pressed glucose plug, formed or positioned within the exit orifice. Regardless of its specific form, the closure will typically comprise a material impermeable to the passage of the active agent formulation, at least until after administration of the dosage form. Suitable closure materials include high-density polyolefin, aluminized polyethylene, rubber, silicon, nylon, synthetic fluorine Teflon®, chlorinated hydrocarbon polyolefins, and fluorinated vinyl polymers.

An exit orifice included in a dosage form of the present invention may also include more than a simple aperture, where desired, the exit orifice may include, for example, a porous element, porous overlay, porous insert, hollow fiber, capillary tube, microporous insert, or microporous overlay. Moreover, regardless of the particular structure providing the exit orifice, a dosage form of the present invention can be manufactured with two or more exit orifices for delivering the active agent formulation during operation. Descriptions of exit orifices suitable for use in controlled release dosage forms are disclosed, for example, in those patents and patent applications already incorporated herein by reference, as well as in U.S. Pat. Nos. 3,845,770, 3,916,899, and 4,200,098, the contents of which are herein incorporated in their entirety by reference.

Though an exit orifice 70 formed of an aperture 72 is only one of various different exit orifices that may be provided in a dosage form 10 of the present invention, exit orifices that are formed as shown in the illustrated embodiments are desirable, as they do not require complete penetration of the reservoir 30 before the dosage form 10 is administered. Such a design works to reduce the possibility that the active agent formulation 40 may leak from the dosage form 10 before the dosage form 10 is administered. Moreover, the aperture 72 included in the exit orifices 70 shown in FIG. 1 through FIG. 6 can be simply formed using known mechanical or laser drilling techniques.

In another aspect, the present invention is directed to a method of manufacturing a dosage form providing the controlled release of an active agent formulation. The method of the present invention includes providing a reservoir including an opening, providing an engine, positioning the engine within the opening of the reservoir and bonding the engine to the reservoir. The method of the present invention also includes loading an active agent formulation into the reservoir, and configuring the dosage form such that an exit orifice is included or formed in the reservoir to allow delivery of the active agent formulation. Though the active agent is preferably loaded before the engine is positioned within and bonded to the reservoir, loading the active agent formulation in the dosage form of the present invention may also take place after the engine and reservoir have been operatively associated.

The step of providing a reservoir including an opening may include providing any reservoir suitable for use in a dosage form of the present invention. For example, the reservoir provided in a method of the present invention may be formed of a water permeable or a water impermeable material, such as those materials disclosed herein. Moreover, the reservoir provided in a method of the present invention may be formed of a single layer of material or multiple layers of one or more different materials. The precise nature of the reservoir provided in a method according to the present invention will depend on, among other factors, the desired application and performance characteristics of the dosage form produced, as well as the nature of the engine and the active agent formulation to be included in the dosage form.

Engines suitable for use in the method of the present invention include any engine that may be used to fabricate a dosage form according to the present invention. For example, the engine may be an osmotic engine or other expandable formulation, device or system. Where the engine provided in the method of the present invention is an osmotic engine, the engine may include a barrier layer and may be formulated or configured to be resistant to permeation by the active agent formulation loaded in the reservoir. However, where the engine provided in a method of the present invention is an osmotic engine 21 that includes a barrier layer, the method of the present invention includes orienting the engine before the engine is positioned within the reservoir such that the barrier layer faces the active agent formulation in the completed dosage form. The precise nature of the engine provided in a method according to the present invention will depend on, among other factors, the desired application and performance characteristics of the dosage form produced, as well as the nature of the reservoir and the active agent formulation to be included in the dosage form.

The step of positioning the engine within the opening included in the reservoir can be carried out using any technique, device or mechanism that results in the desired positioning of the engine within the opening of the reservoir. For example, the positioning step may be carried out by an inserter providing insertion depth control or insertion force control. Preferably, an inserter providing insertion depth control is used to position the engine within the reservoir that has not already been loaded with an active agent formulation, while an inserter providing insertion force control is preferably used to position an engine within a reservoir that has been pre-loaded with an active agent formulation.

Loading the active agent formulation into the reservoir can also be carrier out by any technique, device or mechanism that results in the loading of a desired amount of active agent formulation in the reservoir. Where loading of the active agent takes place before the engine is positioned within the opening of the reservoir, the active agent formulation may be loaded through the same opening used for positioning the engine. However, where the active agent formulation is loaded into the reservoir after positioning the osmotic engine, loading of the active agent formulation must be done either through a second opening formed in the reservoir or by passing the active agent formulation around the engine and into the reservoir. The active agent formulation loaded into the reservoir in a method according to the present invention may be any active agent formulation suitable for use in a dosage form according to the present invention.

The step of configuring the dosage form such that an exit orifice is included or formed in the reservoir may include forming one or more exit orifices as already described herein. For example, the method of the present invention may include creating one or more exit orifices that include a porous element, a porous overlay, a porous insert, a hollow fiber, a capillary tube, microporous insert, or microporous overlay, an aperture or an aperture with a closure, such as a layer of material positioned over the closure, an impermeable bung, cork, or plug, an erodible element, such as a gelatin plug or pressed glucose plug, formed or positioned within the aperture. Moreover, regardless of the particular structure providing the exit orifice, configuring the dosage form such that an exit orifice is included or formed in the reservoir may involve forming two or more exit orifices for delivering the active agent formulation during operation.

In one embodiment, the method of the present invention includes bonding the engine to the reservoir using a bonding material suitable for use in a dosage form according to the present invention. In such an embodiment, the step of bonding may include applying an adhesive or solvent as already described herein to an inside surface of the reservoir, an outside surface of the engine, or to both prior to positioning the engine within the opening of the reservoir. Alternatively, depending on the method used to apply the solvent or adhesive, the step of bonding may include applying a solvent or adhesive to an inside surface of the reservoir, an outside surface of the engine, or to both simultaneously with the step of positioning the engine within the opening of the reservoir.

Were the step of bonding the engine to the reservoir includes the application of an adhesive or a solvent, bonding the engine to the reservoir may also take place after the engine has been positioned within the opening of the reservoir. In such an embodiment, the solvent or adhesive may be introduced into the interstitial spaces formed between an inside surface of the reservoir and an outside surface of the engine either through a passive mechanism, such as capillary action, or by forced introduction, such as by injection or by application of the solvent or adhesive in an environment pressurized to above atmospheric pressure.

In a further embodiment, the method of the present invention includes bonding the engine to the reservoir using a heat sealing technique. In such an embodiment, the reservoir, the engine, or both the reservoir and engine are prepared to include heat responsive material that forms a bond between the engine and reservoir as heat is applied. Suitable heat sealing techniques that may be used to bond the engine of a dosage form of the present invention to the reservoir of the dosage form include, but are not limited to, known tack, spot, or laser welding techniques, hot wheel techniques, or a heat facilitated crimping or clamping techniques.

In yet another embodiment of the method of the present invention, the step of providing an engine includes providing an osmotic engine. Where the engine provided in the method of the present invention is an osmotic engine, the method of the present invention also includes providing a rate controlling membrane. Typically, the step of providing a rate controlling membrane includes providing a rate controlling membrane over at least the portion of the osmotic engine that is not encapsulated by the reservoir. Alternatively, depending on the type of material used to form the reservoir, the step of providing a rate controlling membrane may also include providing a rate controlling membrane over both the exposed portion of the osmotic engine and the reservoir. Where required, providing a rate controlling membrane can be carried out using any materials or methods suitable for creating a rate controlling useful in a dosage form according to the present invention. Particular examples of material and methods for providing a rate controlling membrane include, but are not limited to, those materials and methods described in U.S. Pat. Nos. 6,174,547, 6,245,357 and 4,077,407, U.S. Patent Publication Nos. U.S. 2003-0198619 A1, U.S. 2003-0232078 A1, U.S. 2002-0071863A1, PCT Publications numbered WO 95/34285, WO 04/002448 and WO 01/41742, and U.S. Patent Application No. 60/492,002 (PCT/US04/24921), and Encyclopedia of Polymer Science and Technology, Vol. 3, pages 325 to 354, 1964, published by Interscience Publishers, Inc., New York, the contents which are incorporated in their entirety herein by reference.

### EXAMPLE 1

In order to assess the potential advantages of fabricating a controlled release active agent dosage form having an engine bonded to the reservoir containing the active agent formulation, different exemplary intermediate osmotic dosage forms were fabricated. The first intermediate dosage forms included an engine bonded to a reservoir according to the present invention, while the second intermediate dosage forms included an engine positioned within but not bonded to a reservoir. After fabrication, the mechanical characteristics of the intermediate dosage forms were evaluated to determine the force required to expel the engine from the two different designs.

Both the first intermediate dosage forms and the second intermediate dosage forms were fabricated using the same engines, the same reservoirs, and the same active agent formulation. The active agent formulation was a solution including 5% micronized acetaminophen dissolved in Cremophor EL. The reservoir included in the intermediate dosage forms was provided using clear, size-0 HPMC Vcaps™ capsules supplied by Capsugel®, with the reservoirs being formed only by the capsule bodies of the Vcaps™ capsules. The engines were osmotic engines formed of a bilayer tablet that included an expandable osmotic composition and a barrier layer. The engines were rendered permeation resistant by coating with an HPMC coating.

The bilayer tablet used in each of the engines was manufactured using standard granulation and tableting techniques. The expandable osmotic composition was formulated by first sizing and screening NaCl using a 21-mesh screen and a Quardo Mill set at the maximum speed. Once the NaCl was sized and screened the following dry ingredients were added to and blended in a granulator bowl: 73.70 wt % polyethylene oxide 303, 20.00 wt % NaCl, and 1.00 wt % iron oxide green. In a separate container, a granulating solution was prepared by dissolving 5.00 wt % PVP K29 in purified water. The blended dry ingredients were fluidized in a Glatt Fluid Bed Granulator, and the granulating solution was sprayed onto the fluidized dry ingredients until all of the solution was applied and a granular composition was formed. 0.25 wt % stearic acid and 0.05 wt % BHT were blended with the granular composition to provide an expandable osmotic composition ready for tableting. Two hundred and fifty milligrams of the granular expandable osmotic composition were added to 0.71 cm punch (modified ball lower punch and modified upper punch) and tamped to provide the tableted expandable osmotic composition portion of the bilayer tablet.

The barrier layer composition was also granulated using a Glatt FBG. To prepare the barrier layer composition, Microfine wax and Kolidone SR were blended in a granulator bowl. In a separate container, a granulating solution was prepared by dissolving PVP 29 into purified water. The blended Microfine wax and Kolidone SR were fluidized in the Glatt FBG and the granulating solution was sprayed onto the fluidized constituents until all of the solution was applied and a granular composition was formed. The granulated barrier layer composition included 45.87 wt % Microfine wax, 45.87 wt % Kolidone SR, and 8.26 wt % PVP K29. After the 250 mg of the expandable osmotic composition had been added to the 0.71 cm punch and tamped, 100 mg of the granulated barrier layer composition was added to the punch. The tamped expandable osmotic composition and the barrier layer composition were then compressed using a Korsch press to form a bi-layer tablet including both the expandable osmotic composition and the barrier layer.

To render the bi-layer tablets impermeable to a hydrophobic active agent formulation and complete fabrication of the engines, the bi-layer tablets were coated with an HMPC coating that was resistant to permeation by the active agent formulation. To form the coating aqueous dispersion including 7 wt % of a blend of HPMC 6 cps and PEG 8000 (90/10 w/w ratio) was formed using standard techniques. The aqueous dispersion was then coated onto the bilayer tablets using a standard coating process.

Before the engines were positioned within the reservoirs, 500 mg of the active agent formulation was loaded into the reservoirs using a standard loading process. Once the engines were ready and the reservoirs were loaded with the desired amount of active agent formulation, the first intermediate dosage forms and the second intermediate dosage forms were completed. The engines of the first intermediate dosage forms were bonded to the reservoir by applying a solvent solution formed of 50% ethanol and 50% water at the interface between the outside surface of the engine and the inner surface of the reservoir. Where applied that solvent caused the dissolution of HPMC included in the reservoir and in the coating of the engines, which in turn resulted in a bonding material that fused the engine and the reservoir as the solvent dried. The second intermediate dosage forms were fabricated by simply inserting the coated engines into filled reservoirs using an inserter with insertion force control. The engines included in the second intermediate dosage forms were not bonded to the reservoir.

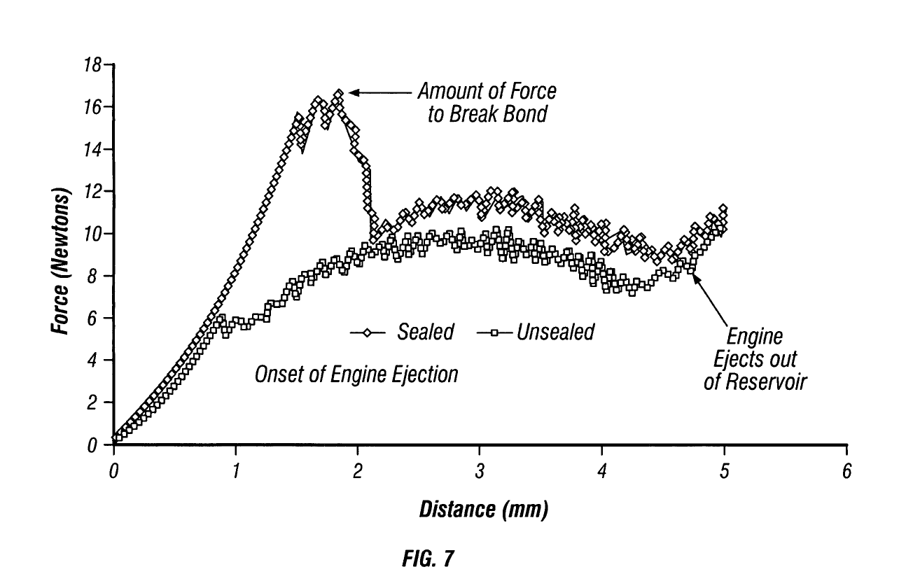

After completion of the intermediate dosage forms, a texture analyzer was used to determine the force required to separate the engine from the filled reservoir of the first and second intermediate dosage forms. The texture analyzer included a metal probe that was positioned against the side of the each intermediate dosage form tested. After placement against the intermediate dosage form being tested, the metal probe slowly exerted a force against the reservoir of the intermediate dosage and the metal probe was stopped when the engine included in the intermediate dosage form separated from the reservoir. Ten different first intermediate dosage forms and ten different second intermediate dosage forms were evaluated, and the results of this evaluation are shown in FIG. 7, which plots the force required to separate the engines included the first intermediate dosage forms as well as the force required to separate the engines included in the second intermediate dosage forms. As can be seen by reference to FIG. 7, a much higher force was required to the cause separation of the engines included in the first intermediate dosage forms than was required to cause separation of the engines included in the second intermediate dosage forms.

### EXAMPLE 2

To evaluate the release rate performance of dosage forms prepared according to the present invention, two groups of controlled release active agent dosage forms were prepared. The two groups of controlled release dosage forms were fabricated from the first and second intermediate dosage forms prepared in Example 1, with the first group of dosage forms being prepared from the first intermediate dosage forms and the second group of dosage forms being prepared from the second intermediate dosage forms.

Completed dosage forms were fabricated by first coating the first and second intermediate dosage forms with rate controlling membranes followed by providing the coated assemblies (including the intermediate dosage forms coated with a rate controlling membrane) with an exit orifice. The rate controlling membrane provided on the first and second intermediate dosage forms assemblies included 90 wt % cellulose acetate 398-10 and 10 wt % Lutrol F-68. The rate controlling membrane was coated on the pre-coating assemblies using a coating solution formed by dissolving the desired amount of cellulose acetate 398-10 and Lutrol F-68 in acetone to provide a coating solution with a solid content of 5 wt %. The coating solution was then spray coated onto the first and second intermediate dosage forms in a 12″ Freud Hi-coater until each of the intermediate dosage forms were coated with about 76 mg of the rate controlling membrane composition. A laser drill was then used to provide each of the coated assemblies with an exit orifice including an aperture having a 20 mil (0.5 mm) diameter formed through the rate controlling membrane. After drilling, the first and second groups of dosage forms were allowed to dry at 45° C. and 45% relative humidity for one day followed by an additional day of drying at 45° C. and ambient relative humidity.

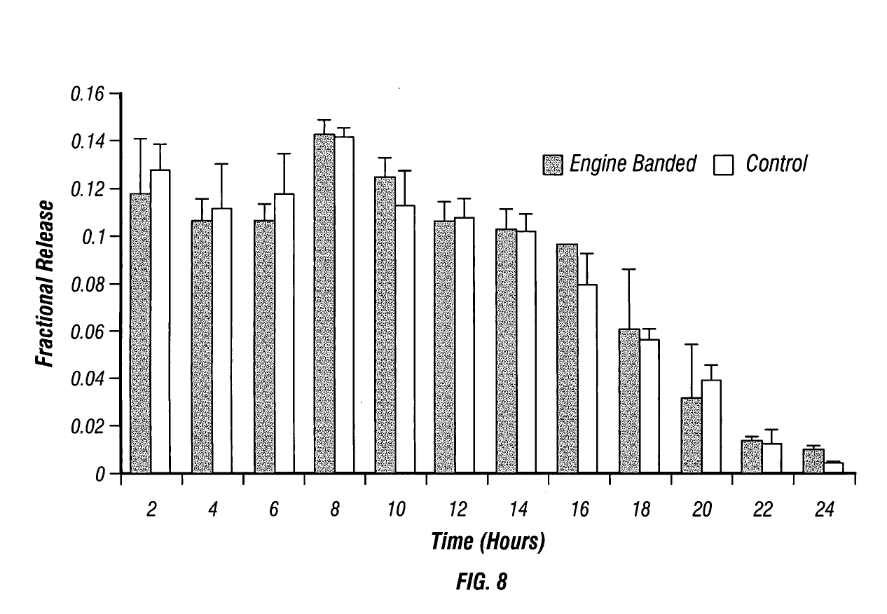

After drying, the release rate profile of acetaminophen provided by the first and second group of dosage forms was measured. Ten dosage forms from both of the first group and the second group were chosen and the release rate profile provided by these dosage forms was measured using a USP VII method in simulated intestinal fluid without enzyme (pH 6.8) at 37° C. The release rate profile of acetaminophen achieved by the first group of dosage forms, which included the engine bonded to the reservoir, can be compared in FIG. 8 to the release rate achieved by the second group of dosage forms. Reference to FIG. 8 shows that release rate functionality of dosage forms from the first group is substantially similar to the release rate functionality achieved by dosage forms from the second group.

1. A dosage form configured to provide the controlled release of an active agent formulation comprising
a reservoir containing an active agent formulation and
an engine partially positioned within the reservoir, and the engine not being completely encapsulated by the reservoir,
wherein the dosage form is configured to expel the active agent formulation from within the reservoir at a controlled rate after administration of the dosage form to an environment of operation.

2. The dosage form of claim 1, wherein the engine is bonded to an inside surface of the reservoir.

3. The dosage form of claim 2 wherein the engine is bonded to the reservoir via an adhesive bond.

4. The dosage form of claim 3, wherein the adhesive bond is formed using an adhesive material applied to an inside surface of the reservoir, to an outer surface of the engine, or to both an inside surface of the reservoir and an outer surface of the engine.

5. The dosage form of claim 2, wherein the engine is bonded to the reservoir via a solvent bond.

6. The dosage form of claim 5, wherein the solvent bond is formed by application of a solvent to an inside surface of the reservoir, to an outer surface of the engine, or to both an inside surface of the reservoir or an outer surface of the engine.

7. The dosage form of claim 2, wherein the engine is bonded to an inside surface of the reservoir via a heat seal bond.

8. The dosage form of claim 5, wherein the heat seal bond is formed using a technique selected from tack or spot welding, laser welding, a hot wheel technique, or a heat facilitated crimping or clamping technique.

9. The dosage form of claim 1, wherein the engine comprises an osmotic engine.

10. The dosage form of claim 9, wherein the osmotic engine comprises an expandable osmotic composition.

11. The dosage form of claim 10, wherein the osmotic engine comprises a barrier layer or an outer coating that limits migration of an active agent formulation from the reservoir into the osmotic engine.

12. The dosage form of claim 1, wherein the reservoir comprises a water permeable material.

13. The dosage form of claim 1, wherein the reservoir comprises a material that is substantially impermeable to water.

14. A dosage form comprising
a reservoir containing an active agent formulation,
an osmotic engine partially positioned within an opening formed within the reservoir, and the osmotic engine not being completely encapsulated by the reservoir,
a rate controlling membrane, and
an exit orifice through which the active agent formulation can be delivered.

15. A method of manufacturing a dosage form providing the controlled release of an active agent formulation comprising:
providing a reservoir having an opening that is sized and shaped to receive an engine,
providing an engine,
positioning the engine within the opening of the reservoir so that the engine partially positioned within the reservoir, and the engine not being completely encapsulated by the reservoir, and
bonding the engine to the reservoir.

16. The method of claim 15 wherein bonding the engine to the reservoir comprises
bonding the engine to the reservoir while the engine is being positioned within the opening of the reservoir

17. The method of claim 15 wherein bonding the engine to the reservoir comprises
bonding the engine to the reservoir after the engine has been positioned within the opening

18. The method of claim 15, further comprising loading an active agent formulation into the reservoir

19. The method of claim 15, further comprising
configuring the dosage form such that an exit orifice is included or formed in the reservoir to allow delivery of the active agent formulation.

20. The method of claim 15, further comprising
bonding the engine to the reservoir using an adhesive.

21. The method of claim 20, wherein bonding the engine to the reservoir using an adhesive comprises applying an adhesive to an inside surface of the reservoir, an outside surface of the engine, or to both prior to positioning the engine within the opening of the reservoir.

22. The method of claim 20, wherein bonding the engine to the reservoir using an adhesive comprises applying an adhesive to an inside surface of the reservoir, an outside surface of the engine, or to both simultaneously with positioning the engine within the opening of the reservoir.

23. The method of claim 16, further comprising:
bonding the engine to the reservoir using a solvent.

24. The method of claim 23, wherein bonding the engine to the reservoir comprises applying a solvent to an inside surface of the reservoir, an outside surface of the engine, or to both prior to positioning the engine within the opening of the reservoir.

25. The method of claim 23, wherein bonding the engine to the reservoir comprises applying a solvent to an inside surface of the reservoir, an outside surface of the engine, or to both simultaneously with the step of positioning the engine within the opening of the reservoir.

26. The method of claim 15, further comprising:
bonding the engine to the reservoir using a heat sealing process.

27. The method of claim 26, wherein the heat sealing process is selected from tack or spot welding, laser welding, a hot wheel technique, or a heat facilitated crimping or clamping technique.

28. The method of claim 16, wherein providing an engine comprises
providing an osmotic engine that comprises a rate controlling membrane

29. The method of claim 28, wherein the rate controlling membrane is formed or positioned over at least a portion of the osmotic engine that is not encapsulated by the reservoir.

30. The method of claim 28, wherein the controlling membrane is formed or positioned over both an exposed portion of the osmotic engine and the reservoir.

31. The method of claim 28, wherein the osmotic engine further comprises a barrier layer

32. The method of claim 31, further comprising:
orienting the osmotic engine before it is positioned within the reservoir such that after the engine is positioned within the opening of the reservoir, the barrier layer faces the active agent formulation.

33. The method of claim 31, wherein the barrier layer comprises a barrier layer that is resistant to permeation by the active agent formulation.
